(19) United States
(12) Patent Application Publication
(10) Pub. No.: US 2001/0043933 A1
(43) Pub. Date: Nov. 22, 2001

(54) Methods and compositions for modulating the immune systems of animals

(76) Inventor: William E. Marshall, Naples, FL (US)

Correspondence Address: ZARLEY MCKEE THOMTE VOORHEES & SEASE PLC, SUITE 3200, 801 GRAND AVENUE, DES MOINES, IA, 50309-2721, US

(21) Appl. No.: 09/883,550

(22) Filed: Jun. 18, 2001

Related U.S. Application Data
(63) Continuation of application No. 09/193,653, filed on Nov. 17, 1998, now abandoned.
Application No. 09/193,653 is a continuation-in-part of application No. 08/739,264, filed on Oct. 29, 1996, now Pat. No. 5,840,318.
Application No. 08/739,264 is a continuation-in-part of application No. 08/517,016, filed on Aug. 18, 1995, now abandoned.
Application No. 08/517,016 is a continuation-in-part of application No. 08/376,175, filed on Jan. 20, 1995, now abandoned.
Application No. 08/376,175 is a continuation-in-part of application No. 08/059,745, filed on May 11, 1993, now abandoned.

(51) Int. Cl. A61K 39/02

(52) U.S. Cl. 424/282.1; 424/234.1

(57) ABSTRACT

Methods and compositions for modulating the immune system of animals by activating macrophages to release cytokines and down-regulating their sensitivity to bacterial endotoxin and to conversion to cytotoxic phenotypes by the administration of high doses of stress response factors released by stressed bacteria. The invention also extends the viability of monocytes to further enhance immunity by administering lower doses of SRFs.

### CROSS REFERENCE TO RELATED APPLICATIONS

This application is a continuation-in-part of application Ser. No. 08/739,264 filed Oct. 29, 1996 now U.S. Pat. No. 5,840,318, which is a continuation-in-part of Ser. No. 08/517,016 filed Oct. 18, 1995 now abandoned, which is a continuation-in-part of Ser. No. 08/376,175 filed Jan. 20, 1995 now abandoned, which is a continuation-in-part of Ser. No. 08/059,745 filed May 11, 1993 now abandoned, the disclosures of which are hereby incorporated by reference.

### FIELD OF THE INVENTION

This invention relates to methods and compositions for modulating immune responses of animals or humans. More particularly, the invention relates to methods of modulating immune responses of animals or humans by administering effective amounts of compositions harvested from stressed bacteria which include stress response factors (SRFs) that activate and modulate circulating macrophages.

### BACKGROUND OF THE INVENTION

Microorganisms commonly encounter threatening changes in their environments. These changes include depletion of nutrients, harmful shifts in temperature and pressure, and sharing habitats with invading organisms. A commonly encountered stress of animal-associated bacteria and one of importance to a study of infectious diseases is that induced through the transfer of cultures from their propagation media into different media. This stress is especially pronounced when the bacteria are transferred to a media with altered or reduced levels of nutrients, such as when exogenous bacteria enter an animal or when endogenous bacteria penetrate sterile zones and tissues from their non-sterile habitats.

Under normal and naturally-occurring stresses, bacteria release products known as stress response factors, (SRFs). These SRFs include polymers of nucleic acids, proteins and peptidoglycans and their partial and complete hydrolysates. Although this release of SRFs can be associated with the deaths of 75-90% of the bacterial population, the released products preserve the viability of the community. The stressors that induce their release are common to commensal bacteria, e.g. over-crowding, diluting, nutrient deprivation and exposure to antibiotics.

Through co-evolution, animal immune systems appear to have adapted responses to the release of bacterial SRFs. This immune reaction is important when SRFs are released by bacteria populating the external surfaces of an animal or human, e.g., the mouth, nose, outer ear, oropharyngeal cavity and vagina. Likewise, bacteria attempting to invade horizontally onto sterile tissue, (e.g. from the outer to the inner ear, from the nose to the sinus, from vagina to uterus), encountering nutrient deprivation will release these factors which serve to alert the host to a potential penetration onto a sterile area or into sterile tissue. This immune response involves the stimulation of activity in monocytes and macrophages.

Specifically, the macrophage has adapted a pre-emptory reaction to the presence of the SRFs that prepares the immune system to defend the host against infection. For example, when commensal bacteria are overcrowded by the presence of growing pathogens, they will release readily absorbable, non-toxic SRFs which activate tissue macrophages to release Interleukin-1, IL-1, Interleukin-6, IL-6 and Tumor Necrosis Factor, alpha, TNFα which stimulate other cells of the immune system. After being highly activated, exposed macrophages down-regulate the surface receptors, CD-14 and CD-16, thereby desensitizing the cell from over-activation by the subsequent interaction with bacterial toxins if infection occurs.

The present inventors have found that the oligomeric fraction having a molecular weight <10 kDa and, in particular, between 500. and 3,000. are readily absorbed, are non-toxic, and both activate and modulate the immune system. The products less than 10 kDa in size are non-toxic and contain further a group of compounds of oligomeric size, i.e. 0.5 to 10 kDa that activate and modulate macrophages. Macrophages are activated to release cytokines at levels deemed helpful to combating infections and are down-modulated to prevent their over-activation with the subsequent release of host-threatening levels of cytokines and becoming overly cytotoxic resulting in perforation of vessels.

As sentry cells, macrophages circulate in the blood and lymph as well as reside in specialized endothelial tissues and organs. They are among the host's first lines of defense, releasing interleukin signals and destroying microbes and diseased cells of the host. Twenty different interleukins can be released by receiving cells, modifying, amplifying, restricting and dampening messages as the system is stimulated. Thus, the macrophage's signal is key to initiating and enforcing the appropriate immune response. In an infection, bacterial endotoxin (lipopolysaccharide, LPS), binds to the CD-14 surface receptor on the macrophage, up-regulating it and inducing the release of yet higher levels of IL-1, IL-6 and TNF. These signals, in turn, induce fever, fatigue, cardiovascular hypotension, renal failure and death in “septic shock”.

To fulfill their role as destroyers of diseased cells of the host, CD-16 recognizes the Fc portion of antibodies. Diseased cells of the host induce the formation of antibodies against presented antigens on their surfaces. The Fc portion of the antibody is received by the CD-16 receptor on the macrophage. Through a complex of reactions, the attached cell is destroyed. By down-regulating the numbers of CD-16 receptors on the surface of the macrophage, the oligomers released by high levels of stressed bacteria help to ensure that the macrophage does not become over-activated by IL-10, thereby destroying healthy host cells. This is the case in septic shock and AIDS, where high numbers of overly active cytotoxic macrophages destroy healthy T-cells.

Monocytes also respond to the release of bacterial SRFs. A high level immune response is induced in monocytes after exposure to SRFs released by bacteria during stress.

Thirteen different species of animal-associated-bacteria have been found to release products when stressed. However, the distribution of polymer:oligomer:monomer is not equal amongst these species. The polymeric fraction (>10 kDa) is toxic when injected into mice, producing a ruffled fur coat, huddling and diarrhea. In vitro assays using human peripheral blood macrophages indicate that the monomeric fraction, (0.5 kDa) does not induce the release of significant levels of interleukins. However, the oligomeric fraction, (between 0.5 and 10. kDa) activates and modulates macrophages, is non-toxic when injected into mice and protected them against a subsequent lethal challenge of injected endotoxin. Therefore, not all strains of bacteria, even of the same species, release levels of oligomers sufficient to protect animals against a subsequent bacterial invasion.

The present inventors have found that the oligomeric stress-response-factors, (SRFs), (between 0.5 and 10. kDa) are a rich new source of natural, normally-occurring, co-evolutionarily evolved immune modulators that can be safely used to protect animals and humans from infections and over-stimulation of their immune system. In addition, this fraction contains compounds that can be used to adjust the expression of individual surface receptors on macrophages to recenter a dysfunctional immune system. Furthermore, in vitro testing indicates their potential role as adjuvants by stimulating the release of IL-12.

An additional discovery is the finding that feral colonies of bacteria yield more oligomeric SRFs when initially stressed than non-feral or laboratory strains. However, inducing a stress upon the repropagated surviving colonies of a laboratory strain will yield a level of oligomeric SRFs comparable to that occurring when a feral strain was stressed.

The discovery of the release of immune-activating and modulating factors has broad implications to improving the immune response through diets and pharmaceutical preparations for humans and animals. Products, e.g. milk, cheese, yogurts) contain viable bacteria, which, when transferred to the nutrient deprived environment of the mouth release SRFs. If such products were formulated to extend the dwell-time in the mouth, more SRFs would be released, activating and modulating a greater local immune response.

Numerous patents teach the healthful benefits of administering specific viable bacteria to humans and animals either orally or parenterally to provide local immune stimulation. Additionally, the prior art recognizes the importance of modulating interleukin release but does not teach the use of safe, natural, normally-occurring products of co-evolution which are effective when taken orally. However, the present invention teaches the administration of sterile, stable, controlled doses of the active principle, oligomeric SRFs, rather than unstable, viable microorganisms.

U.S. Pat. No. 4,975,467 teaches methods by which synthetic compounds can be used to inhibit the release of IL-1 thereby alleviating the induction of its pathophysiologic conditions. U.S. Pat. No. 5,055,447 provides methods and compositions for the prevention of septic shock by administering growth factor-β. This patent teaches the use of administering a signal compound to intercept or modify existing signals. U.S. Pat. Nos. 5,041,427 and 5,158,939 teach the use of a non-toxic LPS from R. spaeroides, ATCC 17023 to desensitize macrophages to toxic LPS. Since R. spaeroides has an unusual lipid A structure, it may not be effective as a desensitizing agent. U.S. Pat. No. 5,157,039 supports the clinical importance for controlling IL-1 release by macrophages by teaching the use of two non-natural quinolinol compounds which appear to be non-selective in IL inhibition.

Further, bacteria release SRFs to help insure the survival of 10 to 25% of their culture. During the aforementioned stress, 70 to 90% of the bacterial population may lose viability. The viability of the survivors is maintained by the presence of SRFs. A further application of this invention is therefore, the use of SRFs to improve the viable shelf-life of dried, frozen or liquid cultures of commercial importance, e.g. starter cultures for the dairy industry.

Livestock are routinely fed silage, a fermented product containing high levels of viable harmless bacteria. When ingested and chewed as cud, the silage bacteria release immune-activating SRFs. The proper selection of harmless bacteria that ferment silage and grains and also release significant levels of SRFs will help improve the health of livestock.

Direct-fed microbials and probiotics are harmless bacteria which are grown in a rich media, concentrated, dried and fed to animals either as a powder for top-dressing or in gel forms for oral inoculation. These products provide a health benefit to the animal in combating infections relating to shipping and weaning. Analysis of commercial products indicates the presence of oligomeric SRFs as well as the release of oligomeric SRFs when transferred to saliva or to a nutrient-reduced environment. It is believed the presence and release of SRFs explains their effectiveness. This discovery permits the administration of a sterile, stable, probiotic of known dose for livestock and poultry. Currently, viability of probiotics is believed necessary for effectiveness. A sterile, stable product allows distribution without refrigeration and would provide a known dose.

The present inventors have now found that the application of short, sequential stresses to the bacteria yield more potent SRFs. Further, they have discovered doses of SRFs that are also effective in rescuing monocytes from apoptosis and, in addition, rescue animals from the lethality of endotoxemia. Moreover, lab tests have been developed which aid in the selection and screening of bacteria which release the more potent SRFs and determining appropriate doses.

It is therefore an object of this invention to provide oral pharmaceutical preparations to help prevent infections in animals.

A further object of this invention is to provide topical pharmaceutical preparations for the activation and modulation of local immune systems to protect against ear, nose and vaginal infections.

Another object of this invention is to provide parenteral injections of individual components of these pharmaceutical preparations to treat septic shock.

An additional object of this invention is to use individual components of these pharmaceutical preparations as adjuvants in conjunction with vaccination.

A further object of this invention is to provide pharmaceutical compositions to down regulate the cytotoxicity of macrophages and prevent their destruction of normal T-cells in persons suffering from HIV infections.

It is yet a further object of this invention to use SRFs as protectants in the storage of starter and other bacterial cultures of use in the food industry.

It is still a further object of this invention to provide SRFs to extend the viability of monocytes, thereby improving their ability to fight infection.

It a further object of this invention to provide screening tests for bacteria which release more potent SRFs.

These and other objectives will become apparent from the following detailed description of the invention.

### BRIEF DESCRIPTION OF THE FIGURES

FIGS. 1, 2, and 3 depict the range of profiles of <10 kDa SRFs released by bacteria when cultures are transferred to an environment with fewer nutrients. The oligomeric fraction that activates and modulates macrophages is that eluted between 10 and 30 ml.

FIG. 1 illustrates the distribution of <10 kDa SRFs released by a non-feral ATCC strain of L. caseii showing a relatively greater amount of non-activating monomers than activating oligomers. However, a higher proportion of oligomeric SRFs are released by the survivors of the initial stress which have been repropagated to 10⁹ CFU/ml and stressed in the same manner, FIG. 2.

FIG. 3 illustrates the <10 kDa SRFs released by the feral and virulent pathogen, L. monocytogenes.

FIG. 4 shows the <10 kDa SRFs released by a direct-fed-microbial or animal probiotic commercially marketed which indicates the presence of macrophage-activating oligomers.

FIG. 5 shows the profile of <10 kDa SRFs released by whole corn silage when transferred to saliva or to 0.01M phosphate buffer, pH 7.5 or in buffer at pH 4., only 25% of the level of SRFs were released indicating that the SRFs resulted from bacterial action rather than from leaching of the plant components.

### SUMMARY OF THE INVENTION

The present invention describes pharmaceutical compositions and methods of use of the same for activating and modulating immune responses in animals. Through the application of a chemical, physical, or biological stress, bacteria produce stress response factors (SRFs). These SRFs are filtered to remove those having a size of 10 kDa or greater. The <10 kDa fraction is then administered to animals to bolster their immune response.

According to a preferred embodiment, the bacteria are subjected to sequential periods of stress for time periods of 20 minutes or less. The bacteria are preferably stressed during the stationary phase of their life cycle, after having been propagated at a temperature between 22-32° C.

The administration of SRFs <10 kDa activate and modulate circulating macrophages and monocytes to stimulate the animal's immune system. The invention also includes screening tests to guide in the selection of bacterial strains and stresses to ensure the release of the most effective levels of immune-stimulating SRFs.

### DETAILED DESCRIPTION OF THE INVENTION

As set forth above, this invention relates to the production of SRFs in bacteria and the administration of these SRFs in animals to modulate their immune response.

As used herein, the phrase “modulating an immune response in animals” includes: (a) stimulating an immune response by activating macrophages to release immune stimulating interleukins IL-1, IL-6 and TNF (for example to prevent or combat infections); (b) down-regulating the CD-14 receptor of macrophages to prevent over-stimulation by endotoxin leading to the over-production of IL-1, IL-6 and TNF, associated with systemic inflammation, cardiovascular dysfunction, shock and death; (c) down-regulating the CD-16 receptor of macrophages to prevent over-stimulation by IL-10 leading to the over-conversion of macrophages to their cytotoxic phenotype with its potential for excessive destruction of host cells, e.g. endothelial cells lining blood vessels and T-cells; and (d) rescuing monocytes from apoptosis.

The composition of the invention may be administered orally, parenterally, topically, or intranasally to stimulate the immune system by: (1) activating macrophages to release cytokines, in particular IL-1, IL-6 and TNF required to initiate an immune response to prevent or reduce infection, (2) by counteracting the potential pathologic role of macrophages in over-stimulating the inflammatory response locally (for example rheumatoid arthritis and other auto-immune diseases) or systemically (for example septic shock), and (3) by rescuing monocytes from apoptosis.

At high levels of the administered compositions, highly expressing macrophages are induced to cell-suicide or apoptosis, thereby ensuring protection against immune dysfunction. Circulating monocytes lose viability after 24 hours via apoptosis when cultured in the absence of a stimulus. The administration of lower levels of SRFs has been found to extend the life of the monocyte population from 24 hours to 72 hours, thereby extending their ability to fight less virulent infections.

The methods of obtaining the composition of the invention comprise growing a selected bacteria in a media outside of the animal to a selected level of enumeration, stressing the selected bacteria thereby initiating the release of stress response factors and thereafter, collecting the supernatant containing the stress response factors. Preferably the stressing of the selected bacteria to induce the release of stress response factors is accomplished by reducing the availability of nutrients to the bacteria. Most preferably this is accomplished by one or more of the following methods after propagating bacteria to the selected level of enumeration. (1) removing-the bacteria from the media by centrifugation and suspending the bacteria in a non-nutritive buffer; (2) adding effective antibiotics to preparations of sensitive bacteria; (3) adding additional bacteria to the media; (4) reducing the volume of the media; (5) removing nutrients from the media; (6) altering the pH of the media to affect the bioavailability of nutrients-from the media; and (7) diluting the bacteria one-thousand fold.

The life cycle of bacteria encompasses a planning phase (lag), a growth phase in which division greatly exceed death (log), a phase in which growth rates approximate death rates (stationary), and a decline phase in which death greatly exceeds growth (death phase). Strains in their death phases are not reliable sources of strong SRFs. Repropagated cultures that have lost 99% of their viability during their death phase are not reliable producers of active SRFs. It has now been found that the method of stressing the bacteria is most preferably performed by removing the bacteria from their media while in their stationary phase (about 10⁸⁻⁹ CFUs per ml) and resuspending them at a 5-fold concentration in a non-nutritive phosphate buffer at pH 7.6 for sequential stresses of periods of 10-20 minutes at 37°-41° C. The non-nutritive phosphate buffer is preferably phosphate buffered saline (PBS) which is a representative animal secretion, e.g. saliva. Even stronger SRFs are produced by growing feral strains to their stationary phase on agar, or in broth at temperatures less than 37° C., between 22-32° C.

In an earlier application, the inventors described the use of periods of stress ranging from 12 to 24 hours. Although bacteria release SRFs immediately when stressed and continue to do so for 12 to 16 hours, it has now been found that shorter period of sequential stresses of 20 minutes or less yield more potent SRFs and SRFs of different potencies. SRFs have been found to be released during periods of stress as short as 10 minutes. The preferred period of stress is about 20 minutes.

Sequential stressing can be performed in a number of ways. A convenient means of sequential stressing is to transfer the bacteria from their growth medium into fresh PBS. This transfer is marked by the immediate release of SRFs. Transferring the bacteria into fresh PBS again induces the release of additional SRFs. A preferred method is to employ endotoxin-free, phosphate-buffer-saline at pH 7.6 in sequential dilutions to mimic physiologic conditions: the dynamic action of bathing and transporting foreign bacteria in body fluids.

The appearance of these factors can be followed by monitoring absorbencies in the ultraviolet, at 220 and preferably at 254 nm. It was discovered that supernatants containing SRFs with a molecular weight greater than 10 kDa were toxic when administered parenterally to mice, so in a preferred embodiment the invention comprises removing all substances greater than 10 kDa by means such as filtration. Thus the supernatant may be filtered so that all SRFs greater than 10 kDa are removed and those of a size less than 10 kDa are retained in solution.

The amount of total SRFs released depends on: (a) the level of bacteria; an optimum level is 5×10⁸ to 3×10¹⁰ CFUs per ml; at higher levels, fewer SRFs are released per cell; (b) the timing of stress; it has now been found that more potent SRFs are produced by transferring bacteria from their stationary phase in rich media into a non-nutritive buffer; (c) strains selected from the wild provide more SRFs than laboratory strains; however, stressing laboratory strains, re-propagating and stressing the survivors yields significant levels of SRFs, (d) the pH of the release solution; pH values below 4.8 induce the release of approximately one-fourth the level of SRFs released at pH 7.6; (e) the temperature; release can be observed at 40° C., and stronger SRFs are produced by bacteria propagated at temperatures less than 37° C., e.g. at 22-32° C.; (f) the time; while release begins immediately, it has-now been discovered that short sequential periods of stress of 20 minutes or less produce more potent SRFs. The molarity and ionic strengths of the releasing solution appear to be of minor significance in the release of SRFs.

Generally, for accumulation of the composition of the invention, the SRF-containing supernatant is rendered bacteria-free by filtering through a 0.22 μm filter to yield a sterile preparation containing all sizes of SRFs. Typically, the total SRF fraction consists of 5-20% polymers larger than 30 kDa, 0.2-20% oligomers between 0.5 and 10 kDa and 0.5-95% monomers less than 0.5 kDa in size. The oligomeric fraction between 0.5 and 10 kDa is non-toxic, readily absorbable, activates macrophages, and extends the viability of monocytes. Daily injections of a protective dose of <10 kDa SRFs for five consecutive days does not cause apparent toxicity in mice, i.e. cessation of eating, ruffled fur, huddling, or diarrhea.

Since the ultraviolet spectrum of the composite of SRFs indicates a maximum of 254 nm, the absorbancy at 254 nm can be used to monitor their release. One Arbitrary Unit (AU) of SRFs was established as that level providing an optical density of 0.001 through 1 cm. of a solution.

SRFs from 15 strains of animal-associated, Gram-positive and Gram-negative, aerobic and anaerobic bacteria representing both harmless and virulent pathogens release SRFs as evidenced by the rise in A254 during stress induced by nutrient reduction. However, the distribution of sizes within 10 to 0.5 kDa was not equal across all strains.

This invention teaches the selection of organisms and the conditions employed to stress them to yield a maximum level of immuno potent SRFs, preferably between 0.5 and 3 kDa.

This invention teaches the improvement of two natural conditions: the consumption of food by humans or feedstuffs by livestock which are rich in microbial populations. Pasteurized fresh milk contains less than 10⁴ CFU of bacteria per ml. Fermented dairy products (milk, yogurts, cheeses) typically contain 10⁶⁻⁸ CFUs per ml. of populations of harmless bacteria in stationery phase. When transferred by eating into a nutrient poor environment, the mouth, SRFs are released at corresponding levels. If the dwell-time in the mouth can be increased by gelling or thickening agents being applied to the food, the release of SRFs and delivery to oropharyngeal macrophages can be increased to immune-stimulating levels. It is this which the inventors believe explains the frequently reported observations regarding immune stimulation and the benefits derived from consuming fermented foods. Fresh vegetables containing high levels of harmless bacteria will also stimulate local macrophages and extend the viability of monocytes by releasing SRFs during eating.

Similarly, the practice of administering oral preparations of 10⁸⁻¹⁰ CFUs of harmless viable bacteria to livestock to reduce the incidence of infections during shipping and weaning results in the release of SRFs by bacteria lyophilized from rich media. Superior products can be formulated by selecting bacteria and the number of sequential stressors that release a predominance of immuno potent SRFs, 0.5 to 3 kDa in sterile formulations that prolong dwell-time in the mouth. In addition, these preparations can contain added SRFs to maintain their viability during storage. Furthermore, probiotic preparations can be delivered bacteria-free by separating and packaging the active fraction, SRFs.

Additionally, bacterial inoculants of 10⁸⁻¹⁰ CFUs of bacteria are commonly added to stored grains and crops to assist and speed the fermentation of plant materials into readily available nutrients for livestock. Presently, strains are selected for their ability to propagate rapidly on the targeted grains and crops. The “probiotic” effect of enhancing the animal's resistance to infection which is often observed from consuming inoculated grains and silages is due to the stimulation of macrophages by SRFs released when these bacteria are introduced into the nutrient-poor environment of the animal's mouth. The probiotic effectiveness of crop inoculants can be increased by selecting strains specific to certain crops plus having the capability to release significant levels of readily-absorbable, non-toxic SRFs as taught herein.

Commercially prepared starter cultures are typically grown to late log phase before centrifuging, washing, resuspending and storing as a refrigerated cake, frozen or freeze-dried powders. Each unit process induces stress, and the marketed culture is the product of sequential stresses containing reduced levels or no SRFs. During storage, viability is lost, but can be maintained by keeping the released SRFs with the cultures or by adding SRFsTotal (both > and <10 kDa) obtained by a surrogate culture that is cheaper to produce or releases more potent SRFsTotal.

The generation of SRFs is not to be confused with the generation of shock proteins resulting from changes in temperature or other conditions. Shock proteins have a molecular weight greater than 10 kDa (typically 30-150 kDa) and their release is not associated with loss of viability. They represent increased synthesis of certain proteins plus the de novo synthesis of new proteins.

The present invention also encompasses a screening test to guide in the selection of strains to produce the most potent SRFs and the conditions for their growth and stress, including the number of sequential stresses to ensure the release of the most effective immune-stimulating SRFs.

The following examples are offered to illustrate but not limit the invention.

### EXAMPLE 1

### Release of SRFs Through Release of A254-Absorbing Compounds

By measuring the release of A254-absorbing compounds, the release of SRFs was observed from approximately 2×10¹⁰ CFU after 10 hours of incubation at 37° C. in 0.01M phosphate buffer, pH 7.5. The average number of Arbitrary Units (AU) per ml +/−50% from three experiments according to Example 2 were as follows:

TABLE 1

| L. acidophilus | 3000.AU/ml |
|---|---|
| L. caseii | 7000. |
| L. fermentum | 3500. |
| L. plantarum | 4000. |
| L. monocytogenes | 24000. |
| S. aureus | 10000. |
| S. typhimurium | 9000. |
| P. acidolactici | 6500. |
| B. coryneforme | 6200. |
| E. coli | 4400. |
| E. faecium | 7000. |
| S. pyogenes | 12000. |
| K. pneumoniae | 8500. |

### EXAMPLE 2

### Production of SRFs from S. aureus

Colonies of S. aureus obtained from a patient, were transferred from agar to 2 ml of Tryptone-Soy-Broth (TSB) and held at 37° C. until turbid, about 4 hours. Thereafter, the volume of TSB was doubled every hour until the absorbancy at 540 nm reached 1.5, corresponding to about 2×10⁹ Colony-Forming-Units/ml. (CFU's) in a volume of 60 ml. The pellet of bacteria was centrifuged at 8000×g for 10 minutes and washed by resuspending in a half-volume of cold saline and centrifuging. The pellet was then suspended in 6 ml of 0.05M phosphate buffer at pH 7.5 containing 0.9% NaCl and incubated at 37° C. for 16 hours. The measured Absorbance at 254 nm (A₂₅₄) immediately began to rise exponentially, reaching a plateau of 12000 after 12 hours.

The supernatant was collected by centrifugation and sterilized by passing through a 0.22μm filter. The <10 kDa SRFs were obtained by passing the sterile supernatant through a filter with a molecular weight cutoff of 10,000. Typically, the A₂₅₄ of the <10 kDa fraction was 75 to 90% of the total supernatant.

### EXAMPLE 3

### Production of SRFs from L. monocytogenes

L. monocytogenes was propagated in Brain-Heart-Infusion (BHI) as described in Example 1 to yield a <10 kDa fraction of SRFs with an A₂₅₄=22. or 22000 Arbitrary Units (AU)/ml.

### EXAMPLE 4

### Production of SRFs from E. coli

E. coli was propagated in Minimal-Media-Davis (MMD) as described in Example 1 to yield a <10 kDa fraction of SRFs with an A₂₅₄=8.000 or 8000 AU/ml.

### EXAMPLE 5

### Production of SRFs from L. caseii

L. caseii was propagated in Mann-Rogosa-Sharpe broth, (MRS) as described in Example 1 to yield a <10 kDa fraction of SRFs with an A₂₅₄=9.250 or 9250 AU/ml after the initial incubation. A second and third serial incubation of 16 hours each to release SRFs yielded 12000 AU/ml and 3250 AU/ml, respectively.

### EXAMPLE 6

### Activation of Macrophages by SRFs

The <10 kDa SRFs from L. caseii were prepared according to Example 5 and were tested for their ability to activate macrophages to release interleukins and down-regulate CD-14 and CD-16 surface receptors by selective deletion of macrophages.

Heparinized peripheral blood was collected from human volunteers and the leukocytes isolated by centrifuging in Ficoll. The buffy coat containing the leukocytes was collected and distributed into wells of a micro-titer plate at a concentration of 10⁵ per well. The macrophages were separated by adherence to the plastic walls of the plate during 4 hours of incubation at 37° C. in a CO₂-rich atmosphere. RPMI 1640 culture media was added providing a final concentration of macrophages of 1 to 3×10⁵/ml. Solutions of phosphate-buffered-saline at pH=7.5 (PBS), or 0.1 M phosphate, pH=7.5 containing SRFs were added in volumes equal to one-tenth the volume of RPMI and incubated as mentioned.

During incubation, aliquots were removed and viable macrophages were counted visually in a hemacytometer. The interleukins IL-1α, IL-6 and TNFα were determined using commercial cytokine kits (R & D Systems, Minneapolis, Minn.). The levels of the surface receptors on the macrophages were determined by adding fluorescent monoclonal antibodies specific for CD-14 and CD-16 and measuring fluorescence in a FACScan flow cytometer. Data were analyzed by the Lysis-1 program.

TABLE 2

| | Interleukins (pg/ml) | | | Surface Receptors* | |
|---|---|---|---|---|---|
| <10 kDa SRFs | IL-1α | IL-6 | TNFα | CD-14 | CD-16 |
| Control: | 0 | 0 | 0 | 70 | 70 |
| Total: | | | | | |
| 800 AU/ml | 25 | 35000 | 2250 | 60 | 60 |
| 80 | 15 | 22000 | 1700 | 70 | 70 |
| 8 | 8 | 5000 | 8500 | 70 | 70 |

Oligomeric Fraction G-10:

| | | | | | |
|---|---|---|---|---|---|
| 800 | 550 | 30000 | 2000 | 30 | 30 |
| 80 | 315 | 21000 | 1600 | 50 | 50 |
| 8 | 80 | 5000 | 900 | 60 | 60 |

*percentage of macrophages with highly expressed CD-14 and CD 16.

The levels of the surface receptor on the macrophages were determined by fluorescent antibody techniques in a flow cytometer. A bimodal distribution of fluorescence indicating high and low-expressing macrophages was observed.

The deletion of macrophages were determined by visual counting as taught in Example 3.

TABLE 3

| G-10: Oligomeric SRFS | Deletion of Macrophages |
|---|---|
| 1000 AU/ml | 55% |
| 100 | 15 |
| 10 | 0 |
| none | 0 |

### EXAMPLE 7

### Prevention of Septic Shock in Mice—4800 AU of SRFs, i.p.

The <10 kDa SRFs from L. monocytogenes were prepared according to Example 3 and were injected into mice to protect them against a lethal dose of septic-shock inducing endotoxin.

Five week old female Balb/C mice were injected i.p. with 0.2 ml of the PBS solution containing 4800 AU of <10 kDa SRFs 18 hours before receiving an injection of 400 μg of LPS from E. coli O 188:B7, (Sigma, St. Louis, Mo.). The mice showed no adverse effects from the SRFs but became sick as evidenced by their cessation of feeding, ruffled fur and huddling together after the injection of LPS. After 32 to 48 hours they recovered fully. The mice that did not receive a pretreatment of SRFs died within 48 hours after the LPS injection.

TABLE 4

| SRFs Injected Once | Percentage of Survivors |
|---|---|
| Intraperitoneally | After Receiving 400 μg LPS. i.p. |
| 4800 AU | 100% (2/2) |
| 1200 | 85 (6/7) |
| 300 | 8 (1/12) |
| none | 0 (0/10) |

### EXAMPLE 8

### Prevention of Septic Shock in Mice—6000 AU of SRFs, oral.

The <10 kDa SRFs from L. monocytogenes were prepared according to Example 3 and fed to mice to protect them against a lethal dose of septic shock inducing endotoxin, LPS.

Five week old female Balb/C mice consumed 6000 AU of <10 kDa SRFs daily in 4 ml in their drinking water for 3 days before receiving an injection of 400 μg of LPS from E. coli O 188:B7. The mice showed no adverse effects from consuming the SRFs but became sick as evidenced by their cessation of feeding, ruffled fur and huddling together after the injection of LPS. After 32 to 48 hours they recovered fully. The mice that did not consume a pretreatment of SRFs died within 48 hours after the LPS injection.

TABLE 5

| SRFs Consumed Daily | Percentage of Survivors |
|---|---|
| in Drinking Water | After Receiving 400 μg LPS. i.p. |
| 6000 AU | 100% (4/4) |
| 1000 | 75 (3/4) |
| none | 0 (0/10) |

### EXAMPLE 9

### Ability of SRFs to Protect Viability of Bacteria

To demonstrate the ability of SRFs to protect the viability of bacteria, 90000. AU of lyophilized <10 kDa SRFs collected from the total SRFs released by 10 ml of a culture containing S. aureus at 1×10¹⁰ CFU per ml were added to a closed flask containing 100 ml of M. elsdenii, ATCC 25940, at 3×10⁹ CFU per ml. M. elsdenii, an obligate anaerobe-was left in its spent media at 25° C. The addition of SRFs protected the viability of 90% of the M. eldsdenii against oxygen and the toxic effects of its metabolic products.

TABLE 6

| Time | Control | Control + 90000. AU of SRFs |
|---|---|---|
| Day 0 | 2.55 × 10⁹ | 2.55 × 10⁹ |
| Day 2 | 1.25 × 10⁹ | 3.9 × 10⁹ |
| Day 6 | 1.75 × 10⁸ | 1.35 × 10⁹ |

### EXAMPLE 10

### Stress-Inducing Effects of Antibiotics

To demonstrate the stress-inducing effects of antibiotics, penicillin and streptomycin were added at a final concentration of 1% to a culture of E. coli ATCC 11775 growing to mid-log phase of 2.4×10⁸ CFU/ml in shaking Davis Minimal Medium+0.1% dextrose. After 3 hours, aliquots were removed for enumeration and analysis for the generation of SRF.

TABLE 7

| Time | Without Antibiotic | Antibiotic Added |
|---|---|---|
| 3 hours | 6 × 10⁸ CFU/ml | <1 × 10⁷ CFU/ml |
| AU@254 nm | 390. | 630. |

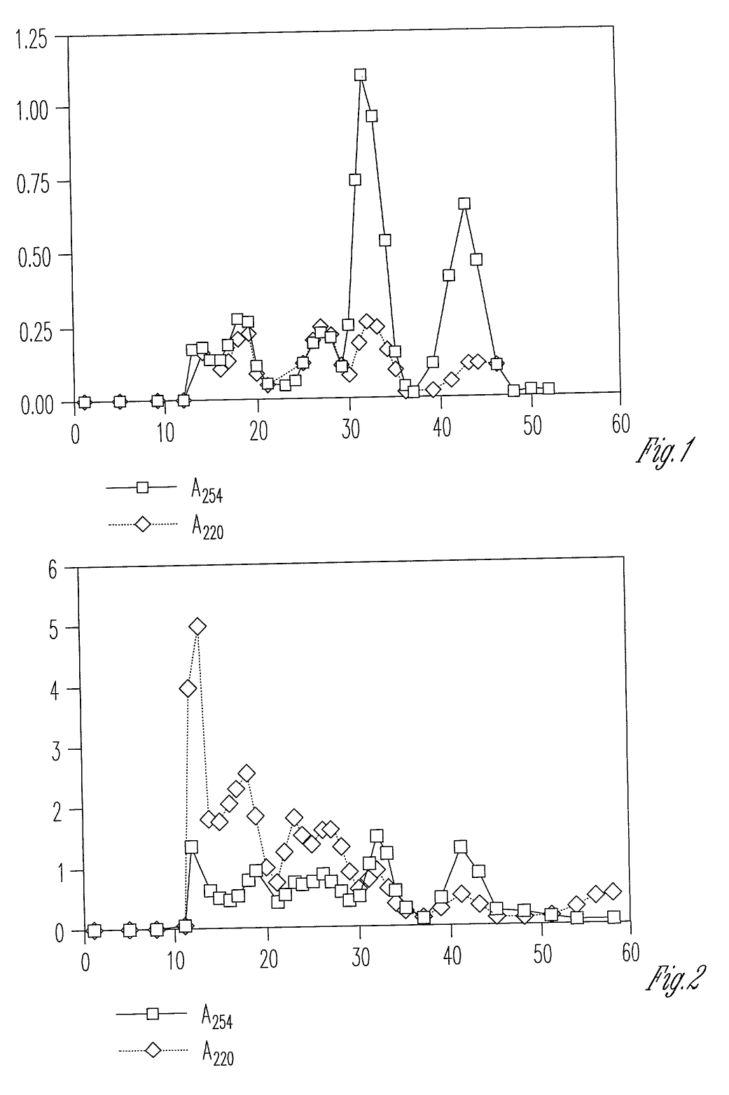

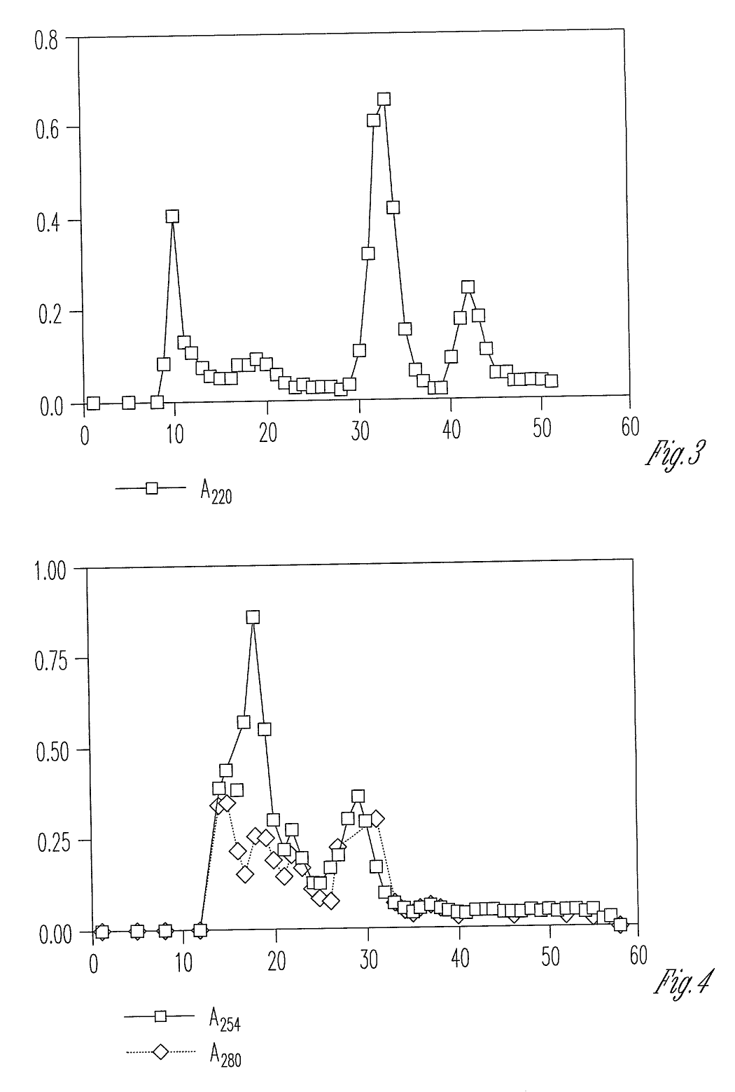

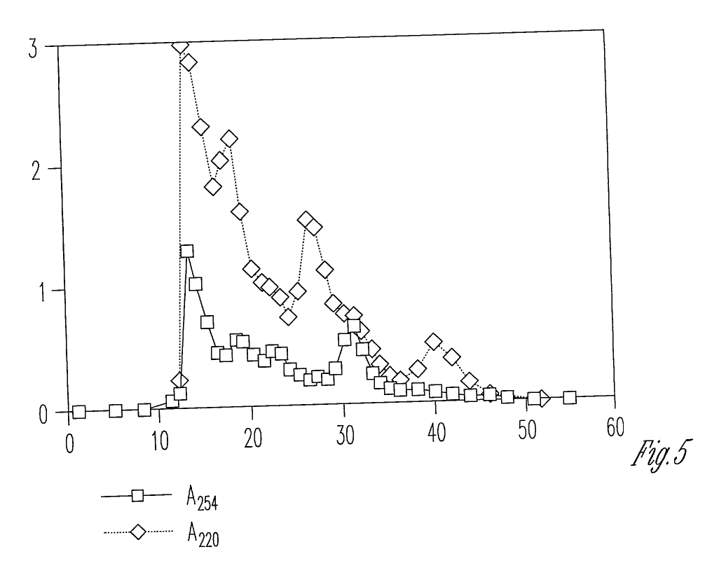

To demonstrate the inducing effects of antibiotics on bacteria in stationery phase, propagating aliquots from the above experiment were allowed to pass into their stationery phase by continuing incubation for another 24 hours. Chromatography on Sephadex G-10 showed a profile similar to those of FIG. 1.

TABLE 8

| Time | Without Antibiotic | Antibiotic Added |
|---|---|---|
| 3 hours | 6 × 10⁸ CFU/ml | 4 × 10⁸ CFU/ml |
| AU@254 nm | 470. | 690. |

### EXAMPLE 11

### Monocyte Assay

Mangan and co-workers demonstrated that human circulating monocytes lose viability after 24 hours via apoptosis (programmed cell death) when cultured in the absence of a stimulus. Mangan, D. F. et al. (1991), “Lipopolysaccharide, Tumor Necrosis Factor-alpha and Interleukin 1B Prevent Programmed Cell Death (apoptosis) in Human Peripheral Blood Monocytes,” J. Immunol, 146:1541. He and co-workers used this dependency to measure the stimulating powers of bacterial components and cytokines. This procedure was adapted to measure the stimulating capacities of SRFs to rescue human monocytes from apoptosis.

Typically, a 10,000-fold dilution of SRFs released by 10⁷⁻⁹ CFUs of bacteria extended the life of 30 to 70% of the monocyte population from 24 to 72 hours (see Table 9). Without activation, fewer than 10% of the cells survived 72 hours. At this-dilution, a negative outcome in the monocyte assay is associated with a negative outcome in the mouse assay, i.e. SRFs that do not activate monocytes will not protect mice.

### EXAMPLE 12

### Mouse Assay

A mouse assay was conducted to measure the potency of monocyte-rescuing SRFs. Feeding 50 μl of strong SRFs<10 kDa released by 10⁷⁻⁹ CFUs of viable bacteria protect mice from the lethality of a subsequent i.p. challenge of a lethal dose of E. coli endotoxin, 300 μg (see Table 9).

### EXAMPLE 13

### Preparation of SRFs from L. plantarum

A single colony of L. plantarum isolated from silage or a leafy vegetable was grown on MRS agar at 30° C. and transferred by loop to 0.5 ml of MRS broth for overnight culturing at 30° C. The volume was increased in increments of 10 volumes of broth added every 12 hours.

The suspension was centrifuged and the pellet resuspended in endotoxin-free, phosphated-buffered saline at pH 7.6 (Dulbecco's PBS Sigma, St. Louis, Mo.) for 20 minutes at 37° C. The suspension was again centrifuged and the pellet resuspended in PBS for a second 20 minute period at 37° C. The supernatants were sterilized by passing through a 0.22 μm filter (Millipore Corp.) and then fractionated through a molecular filter with a cut-off of 10 kDa (Centricon 10, Millipore Corp.) to yield three fractions of SRFs: Total, >10 kDa, and <10 kDa. The SRFs released after the initial PBS stress are referred to as “A”, those from-the second as “B”, and the third “C”. Preparations were frozen at 0° C. until tested (see Table 9).

### EXAMPLE 14

### Preparation of SRFs from L. monocytogenes

A single colony of L. monocytogenes which had been originally isolated from infected cheese was grown on BHI agar, transferred and stressed as in Example 13 (see Table 9).

### EXAMPLE 15

### Preparation of SRFs from Lactobacilli and Enterococci

Colonies of a mixed culture consisting of Lactobacilli and Enterococci were propagated and stressed as described in Example 13 (see Table 9).

### EXAMPLE 16

### Preparation of SRFs from Silage

Three ml of PBS were added to 1 g of corn silage, or whole-plant corn silage or alfalfa silage and treated as in Example 13 (see Table 9).

### EXAMPLE 17

### Measure of Viable Monocytes Following Administration of SRFs

SRFs <10 kDa prepared according to Example 13 were diluted by 5 ten-fold dilutions and 10 μl were added to 90 μl of freshly prepared human circulating monocytes in RPMI 1640 (Sigma) containing 8% fetal bovine serum and incubated at 37° C. as described, (Mangen, D. F. et al.). After 72 hrs the viable monocytes were labeled with a fluorescent anti-CD-14 and measured by flow cytometry in a FACScan. Viable monocytes were reported as a percentage of total monocytes (see Table 9).

### EXAMPLE 18

### Survivability of Mice Injected with SRFs

Fifty μl of SRFs <10 kDa were fed to 25 g female Balb/C mice by means of a micropipette. Forty-eight hours later the mice were injected i.p. with 300 μg of endotoxin prepared from E. coli (Sigma). Control mice received 50 μl of PBS. Survivors were those mice that regained normal health, usually within 5 to 7 days post injection. (See Table 9 below).

TABLE 9
Potencies of SRFs <10 kDa Released by Stressed Bacteria

| Origin of SRFs | Surviving Monocytes | Surviving Mice |
|---|---|---|
| None - control | 7-11% | 0% |
| L. plantarum A | 10 minute stresses | |
| A < 10 | 4% | 0% |
| B < 10 | 26 | 100 |
| C < 10 | 2 | 0 |
| | 20 minute stresses | |
| A < 10 | 10 | 0 |
| B < 10 | 52 | 100 |
| C < 10 | 51 | 0 |
| L. plantarum B | 10 minute stresses | |
| A < 10 | 19 | 0 |
| B < 10 | 5 | 0 |
| L. caseii | 10 minute stresses | |
| A < 10 | 18 | 40 |
| B < 10 | 5 | 0 |
| C < 10 | 4 | nd |
| D < 10 | 61 | nd |
| L. acidophilus | 10 minute stresses | |
| A < 10 | 17 | 0 |
| A < 10 | 12 | 0 |
| C < 10 | 8 | nd |
| L. monocytogenes | 10 minute stresses | |
| A-Total SRFs | 55 | 66 |
| A < 10 | 67 | 100 |
| B < 10 | 9 | 0 |
| | 20 minute stresses | |
| A-Total SRFs | 59 | 100 |
| A < 10 | 46 | 33 |
| B < 10 | 9 | 0 |
| Heat Killed L. | 10 minute stresses | |
| monocytogenes | | |
| A < 10 | 78 | 0 |
| B < 10 | 58 | 0 |
| Uninoculated MRS or | 20 minute stresses | |
| BHI Culture | | |
| A < 10 | 6 | 0 |
| B < 10 | 8 | 0 |
| Uninoculated Corn | 10 minute stresses | |
| Silage | | |
| A < 10 | 11 | 0 |
| B < 10 | 54 | 0 |
| Commercial Silage | 20 minute stresses | |
| Inoculant | | |
| A < 10 | 10 | 0 |
| B < 10 | 51 | 50 |

As can be seen from the above examples, the invention accomplishes its stated objectives. of course certain variations from what has been illustrated and described can be made without departing from the spirit and scope of the invention and those are intended to be encompassed either literally or by the doctrine of equivalents.

What is claimed is:

1. A method for activating and modulating the immune system of an animal comprising:
growing bacteria in a medium;
exposing said bacteria to biological, chemical or physical stress for at least one period of time of 20 minutes or less so that the bacteria release a stress response product;
separating said medium and stress response product from said bacteria to form a separated product;
filtering said separated product to remove any stress response products having a molecular weight of greater than 10 kDa to form a filtrate;
administering said filtrate to said animal.

2. The method of claim 1 wherein said step of stressing comprises reducing the bioavailability of nutrients to said bacteria.

3. The method of claim 2 wherein the bioavailability of nutrients is reduced by transferring the bacteria from a nutrient-rich media to a non-nutritive media.

4. The method of claim 3 wherein said non-nutritive media comprises saline.

5. The method of claim 4 wherein said saline media is a phosphate-buffered saline having a pH of about 7.6.

6. The method of claim 1 wherein the bacteria is selected from the group consisting of Lactobacillus, Staphylococcus, Streptococcus, Pediococcus, Pseudomonas, Bacillus, Escherichia, Listeria, Enterococcus, and Klebsiella.

7. The method of claim 6 wherein the bacteria is selected from the group consisting of L. acidophilus, L. caseii, L. fermentum, L. plantarum, L. monocytogenes, S. aureus, S. typhimurium, P. acidolactici, B. coryneforme, E. coli, E. faecium, S. pyogenes, and K. pneumoniae.

8. The method of claim 1 wherein the bacteria are propagated at a temperature of 37° C. or less.

9. The method of claim 8 wherein the temperature is in the range of 22° C. to 32° C.

10. The method of claim 1 wherein the bacteria are exposed to a stress while they are in their stationary phase.

11. The method of claim 1 wherein the filtering step includes:
passing said separated product through a 0.22 μm filter to form a sterilized product; and
passing said sterilized product through a filter with a molecular weight cutoff of 10,000.

12. The method of claim 1 wherein the filtrate containing the SRFs <10 kDa is administered to an animal selected from the group consisting of humans, poultry and livestock.

13. The method of claim 1 wherein the stress response product is administered in a concentration of about 1000 to 50,000 AU of said stress response product/ml.

14. The method of claim 1 wherein the stress response product is administered in a manner selected from the group consisting of orally, topically, and parenterally.

15. The method of claim 1 wherein the animal is administered stress response products having a size of between 0.5 and 3 kDa.

16. The method of claim 1 wherein the stress response products are administered as an adjuvant for oral or parenteral vaccines.

17. The method of claim 1 wherein the bacteria are exposed to sequential periods of stress.

18. The method of claim 17 wherein the bacteria are exposed to sequential periods of stress by transferring the bacteria from growth media into non-nutritive media, then subsequently transferring the bacteria to non-nutritive media sequentially.

19. The method of claim 18 wherein the bacteria is exposed to three sequential periods of stress.

20. An immune modulating composition comprising: bacterial stress response factors, said stress release factors having a size of less than 10 kDa; and
a pharmaceutically acceptable carrier.

21. An assay for measuring the stimulating capacities of stress response factors to rescue human monocytes from apoptosis, wherein said assay comprises a means capable of measuring the percentage of viable monocytes in a sample following exposure to stress release factors.

22. An assay for measuring the potency of stress release factors, wherein said assay comprises a means of determining the percentage of mice that are protected from endotoxins following standardized exposure to stress release factors from various types of organisms.
